(19) United States
(12) Patent Application Publication
(10) Pub. No.: US 2012/0184898 A1
(43) Pub. Date: Jul. 19, 2012

(54) GAS BASED WOUND AND TISSUE THERAPEUTICS

(76) Inventors: Kevin R. Ward, Glen Allen, VA (US); Robert Diegelmann, Richmond, VA (US); Bruce Spiess, Manakin-Sabot, VA (US); Gary S. Huvard, Chestrefield, VA (US); Gurbhagat S. Sandhu, Manakin-Sabot, VA (US); Gary Bowlin, Mechanicesville, VA (US); Chris Stern, Raleigh, NC (US)

(21) Appl. No.: 13/433,120

(22) Filed: Mar. 28, 2012

Related U.S. Application Data
(63) Continuation of application No. 12/378,460, filed on Feb. 13, 2009.
(60) Provisional application No. 61/198,933, filed on Nov. 12, 2008.
(60) Provisional application No. 61/192,535, filed on Sep. 19, 2008.
(60) Provisional application No. 61/028,230, filed on Feb. 13, 2008.

(51) Int. Cl. A61M 37/00

(52) U.S. Cl. 604/24

(57) ABSTRACT

This invention provides articles of manufacture and bandages comprising compartments and layers comprising oxygen and other therapeutic gas storage forms and perfluorocarbons. This invention also provides for methods of delivering oxygen and other therapeutic gases to a tissue in a subject comprising a administering to the tissue a composition comprising a perfluorocarbon and a oxygen or therapeutic gas storage form, so as to thereby deliver oxygen or the therapeutic gas to the tissue.

This application claims the benefit of U.S. Provisional Application No. 61/198,933, filed Nov. 12, 2008, U.S. Provisional Application No. 61/192,535, filed Sep. 19, 2008 and U.S. Provisional Application No. 61/028,230, filed Feb. 13, 2008, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Traditional treatment of wounds, such as burns, chronic skin ulcers and the like has relied on ensuring proper intravascular resuscitation to help ensure adequate perfusion of the wounds to maintain levels of oxygen in the wound capable of meeting tissue needs and thus promote survival and healing. When perfusion is severely jeopardized, for example in cases of chronic wounds such as pressure, diabetic, venous stasis and arterial ulcers where the vasculature has been severely damaged over time, the use of hyperbaric oxygen has been advocated as a means to enhance oxygenation. In addition to being bactericidal and static, intermittent use of hyper-oxygenation is believed to be beneficial as it may lead to increased tissue oxygenation and ultimate angiogenesis. Furthermore, increased tissue oxygenation may assist in decreasing the inflammatory response. However, use of hyperbaric oxygen and local oxygen applications are expensive, have time constraints, and are labor intensive.

There is a need for a convenient and inexpensive method to deliver high levels of therapeutic gases, such as oxygen to wounds and other tissues.

### SUMMARY OF THE INVENTION

The subject application provides for an article of manufacture comprising a first compartment comprising an oxygen storage form and a second compartment comprising a perfluorocarbon, wherein the first and the second compartments are separated by a gas-permeable and liquid-impermeable material.

The subject application also provides for an article of manufacture comprising a therapeutic gas storage form and a perfluorocarbon, wherein the therapeutic gas is nitric oxide, carbon monoxide, carbon dioxide, or hydrogen sulfide.

The subject application provides for a bandage comprising: a) a substantially gas-tight cover; b) a first layer comprising a therapeutic gas storage form; c) a membrane; d) a second layer comprising a perfluorocarbon; and e) a rayon mesh, wherein the membrane separates the first and second layers.

The subject application also provides for a bandage comprising: a) a substantially gas-permeable cover; b) a layer comprising a perfluorocarbon; and c) a rayon mesh.

The subject application also provides for a method of delivering oxygen to a tissue in a subject comprising administering to the tissue a composition comprising a perfluorocarbon and an oxygen storage form, wherein the perfluorocarbon and the oxygen storage form are separated by a gas-permeable and liquid-impermeable material, so as to thereby deliver oxygen to the tissue.

The subject application also provides for a method of delivering oxygen to a tissue in a subject comprising topically administering to the tissue a solid or semi-solid composition comprising a perfluorocarbon and an oxygen storage form, wherein the perfluorocarbon and the oxygen storage form are separated by gas-permeable and liquid-impermeable material, so as to thereby deliver oxygen to the tissue.

The subject application also provides for a method of delivering a therapeutic gas to a tissue in a subject comprising administering to the tissue a composition comprising a perfluorocarbon and a therapeutic gas storage form, wherein the therapeutic gas is nitric oxide, carbon monoxide, carbon dioxide, or hydrogen sulfide, so as to thereby deliver the therapeutic gas to the tissue.

The subject application also provides for a method of delivering a therapeutic gas to a tissue in a subject comprising topically administering to the tissue a solid or semi-solid composition comprising a perfluorocarbon and the therapeutic gas storage form, wherein the therapeutic gas is nitric oxide, carbon monoxide, carbon dioxide, or hydrogen sulfide, so as to thereby deliver the therapeutic gas to the tissue.

### BRIEF DESCRIPTION OF THE DRAWINGS

FIG. 1 shows the sequence of events of oxygen production from urea hydrogen peroxide.

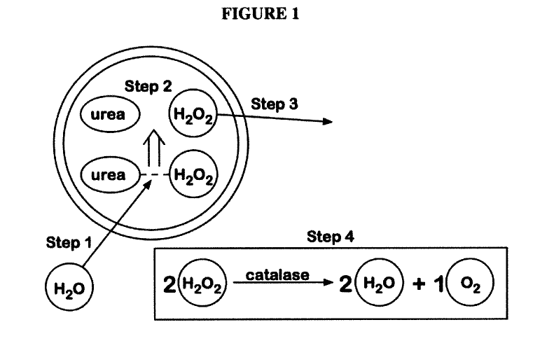

FIG. 2 shows the cross section of one embodiment of the present invention.

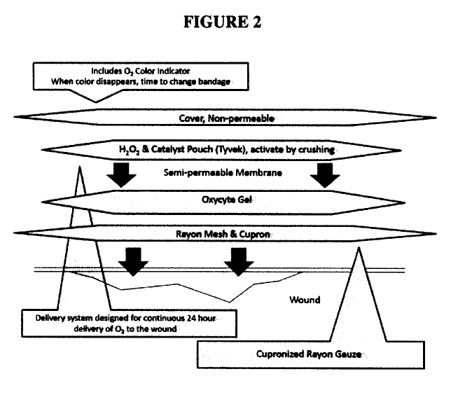

FIG. 3 shows one embodiment of the present invention wherein an O₂ generator is on a hinge attached to a one-piece foil seal.

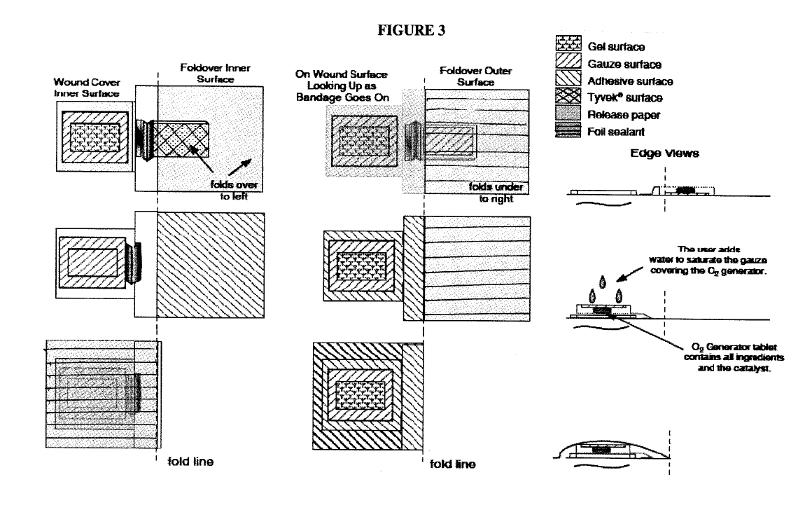

FIG. 4 shows one embodiment of the present invention wherein an O₂ generator is fixed in position with a cover flap.

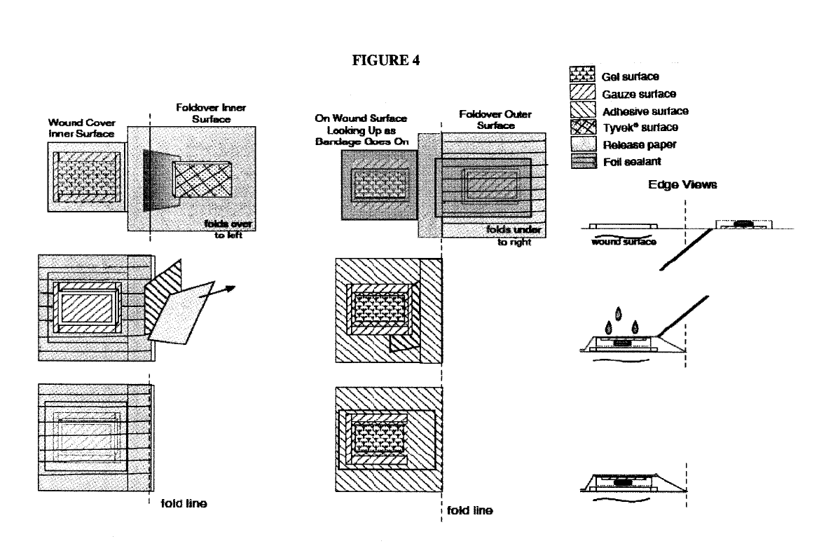

FIG. 5 shows one embodiment of the present invention wherein a bandage comprises vertically stacked gel, O₂ generator and pull-out separator.

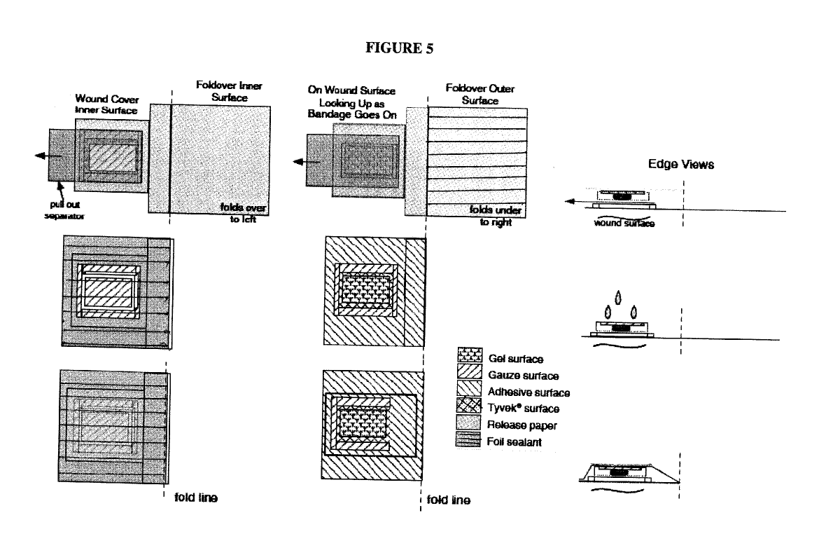

FIG. 6 shows one embodiment of the present invention wherein a perfluorocarbon is applied directly to the wound and the wound is covered by a catalytic oxygen generator followed by an impermeable surface.

FIG. 7 shows a graph of oxygen release rate (g O₂/min) from a bandage according to the present invention.

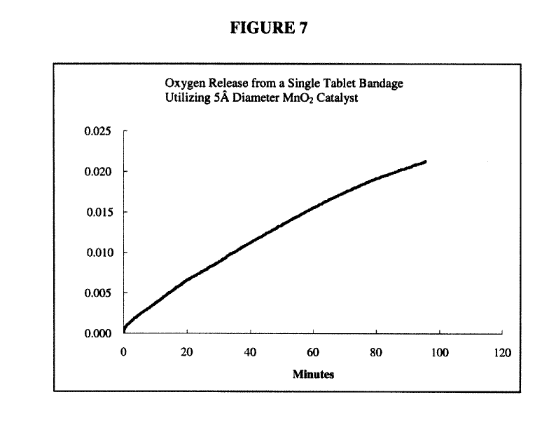

### DETAILED DESCRIPTION OF THE INVENTION

The subject application provides for an article of manufacture comprising a first compartment comprising an oxygen storage form and a second compartment comprising a perfluorocarbon, wherein the first and the second compartments are separated by a gas-permeable and liquid-impermeable material.

In one embodiment, the oxygen storage form is sodium peroxide, calcium peroxide, magnesium peroxide, zinc peroxide, lithium peroxide, urea hydrogen peroxide, sodium percarbonate, sodium percarbonate perhydrate, sodium carbonate perhydrate, sodium perborate, carbamide peroxide, histidine hydrogen peroxide, adenine hydrogen peroxide, anhydrous poly(vinyl pyrrolidone)/hydrogen peroxide complex, or alkaline peroxyhydrate including sodium orthopohsporate. In a preferred embodiment, the oxygen storage form is hydrogen peroxide.

In one embodiment, the perfluorocarbon is perfluoro-tert-butylcyclohexane. In a preferred embodiment, the perfluorocarbon is Oxycyte®.

In one embodiment, the gas-permeable and liquid-impermeable material exhibits at least 10 inches of hydro-head. In another embodiment, the gas-permeable and liquid-impermeable material exhibits at least 30 inches of hydro-head. In yet another embodiment, the gas-permeable and liquid-impermeable material is composite fabric, spundbond-meltblown-spundbond fabric, spundbond-meltblown-spundbond laminate, hydrophobic coated paper, hydrophobic coated fabric, fiberglass filter, microporous polymer membrane, microporous sintered metal membrane, thermo-mechanically expanded polytetrafluoroethylene (PTFE), fluoropolymer, flashspun high-density polyethylene fiber, poly(glycolic acid), poly(vinyl alcohol), polymer coated fabric, fluoropolymer-based porous membrane, or polyurethane-coated fabric. In another embodiment, the gas-permeable and liquid-impermeable material is Gore-Tex®. In another embodiment, the gas-permeable and liquid-impermeable material is Teflon®-based porous membrane. In a preferred embodiment, the gas-permeable and liquid-impermeable material is Tyvek®.

In one embodiment, the first compartment further comprises a catalyst for releasing oxygen from of the oxygen storage form. In another embodiment, the catalyst is a metal or metal alloy containing iron, copper, lead, platinum, silver, iodine, or mercury. In another embodiment, the metal catalyst is a metal oxide or metal salt including manganese dioxide, manganous oxide, titanium dioxide, ferric oxide, ferrous oxide, iron chloride, hydroxide of lead, silver cobalt, manganese, osmium, copper, nickel, iron, chromium, selenium and platinum. In another embodiment, the catalyst is an enzyme including catalase. In yet another embodiment, the catalyst is a hydrogen peroxide oxidizer including iodine, ferric iron compound, mercury compound, silver compound, inorganic nitrate, bromine, concentrated sulfuric acid, chlorine gas, chromate compound, permanganate compound, ozone, and fluorine. In one embodiment, the catalyst provides control release of oxygen.

In one embodiment, the second compartment further comprises a biologically active agent. In another embodiment, the biologically active agent is an antibacterial agent, copper oxide, antibiotic, phospholipid, collagen particle, anti-inflammatory agent, tissue regenerating compound, blood thinner, blood coagulant, pain reliever, anti-itch compound, anti-burn compound, analgesics, matrix metallo proteinase inhibitor, tetracycline, doxycycline, denatured collagen, gelatin, oxidized regenerated cellulose, honey, antifungal compound, NIMBUS antimicrobial compound, calcium alginate, hemostatic agent, protease inhibitor with broad specificity for the inhibition of serine, cysteine, aspartic proteases and amino peptidases, HIV, protein synthesis inhibitor, puromycin, anisomycin, Glyco Pore, Heta starch, alpha-1 proteinase inhibitor human, alpha-2-macroglobulin, tissue inhibitor of metalloproteinases, hyaluronic acid, glycoaminoglycan, proteoglycan, enzymatic debridement agent, bacterial collagenase, chemical or biogenerator for nitrous oxide, carbon dioxide, hydrogen sulfide or other therapeutic gases.

In one embodiment, the biologically active agent is a growth factor or cytokine. In another embodiment, the growth factor or cytokine is PDGF, KGF-2, TGF-β, bFGF, GM-CSF, heparin-binding growth factor-1, TGFα, VEGF, HIF-1, FGF, or CTGF.

In one embodiment, the biologically active agent is a protease inhibitor. In another embodiment, the protease inhibitor is amprenavir, fosamprenavir, indinavir, lopinavir, ritonavir, saquinavir, or nelfinavir.

In one embodiment, the oxygen storage form is in the form of a tablet. In another embodiment, the article of manufacture further comprises a source of water. In another embodiment, the article of manufacture further comprises a second therapeutic gas. In yet another embodiment, the article of manufacture further comprises an oxygen color indicator.

The subject application also provides for an article of manufacture comprising a therapeutic gas storage form and a perfluorocarbon, wherein the therapeutic gas is nitric oxide, carbon monoxide, carbon dioxide, or hydrogen sulfide.

In one embodiment, the therapeutic gas storage form is in a first compartment and the perfluorocarbon is in a second compartment. In another embodiment, the perfluorocarbon is perfluoro-tert-butylcyclohexane. In a preferred embodiment, the perfluorocarbon is Oxycyte®.

In one embodiment, the first and second compartments are separated by a gas-permeable and liquid-impermeable material. In another embodiment, the gas-permeable and liquid-impermeable material exhibits at least 10 inches of hydro-head. In another embodiment, the gas-permeable and liquid-impermeable material exhibits at least 30 inches of hydro-head. In yet another embodiment, the gas-permeable and liquid-impermeable material is composite fabric, spundbond-meltblown-spundbond fabric, spundbond-meltblown-spundbond laminate, hydrophobic coated paper, hydrophobic coated fabric, fiberglass filter, microporous polymer membrane, microporous sintered metal membrane, thermo-mechanically expanded polytetrafluoroethylene (PTFE), fluoropolymer, flashspun high-density polyethylene fiber, poly(glycolic acid), polyvinyl alcohol), polymer coated fabric, fluoropolymer-based porous membrane, or polyurethane-coated fabric. In another embodiment, the gas-permeable and liquid-impermeable material is Gore-Tex®. In another embodiment, the gas-permeable and liquid-impermeable material is Teflon®-based porous membrane. In a preferred embodiment, the gas-permeable and liquid-impermeable material is Tyvek®.

In one embodiment, the first compartment further comprises a catalyst for releasing the therapeutic gas from the therapeutic gas storage form. In one embodiment, the catalyst provides control release of the therapeutic gas.

In another embodiment, the instant article of manufacture is adapted to contain other therapeutic gases known to be beneficial by to those of ordinary skill in the art. The storage forms of these gases and the catalysts for releasing said gases from their corresponding storage forms are known to those of ordinary skill in the art.

In one embodiment, the second compartment further comprises a biologically active agent. In another embodiment, the biologically active agent is an antibacterial agent, copper oxide, antibiotic, phospholipid, collagen particle, anti-inflammatory agent, tissue regenerating compound, blood thinner, blood coagulant, pain reliever, anti-itch compound, anti-burn compound, analgesics, matrix metallo proteinase inhibitor, tetracycline, doxycycline, denatured collagen, gelatin, oxidized regenerated cellulose, honey, antifungal compound, NIMBUS antimicrobial compound, calcium alginate, hemostatic agent, protease inhibitor with broad specificity for the inhibition of serine, cysteine, aspartic proteases and amino peptidases, HIV, protein synthesis inhibitor, puromycin, anisomycin, Glyco Pore, Heta starch, alpha-1 proteinase inhibitor human, alpha-2-macroglobulin, tissue inhibitor of metalloproteinases, hyaluronic acid, glycoaminoglycan, proteoglycan, enzymatic debridement agent, bacterial collagenase, chemical or biogenerator for nitrous oxide, carbon dioxide, hydrogen sulfide or other therapeutic gases.

In one embodiment, the biologically active agent is a growth factor or cytokine. In another embodiment, the growth factor or cytokine is PDGF, KGF-2, TGF-β, bFGF, GM-CSF, heparin-binding growth factor-1, TGFα, VEGF, HIF-1, FGF, or CTGF.

In one embodiment, the biologically active agent is a protease inhibitor. In another embodiment, the protease inhibitor is amprenavir, fosamprenavir, indinavir, lopinavir, ritonavir, saquinavir, or nelfinavir.

In one embodiment, the therapeutic gas storage form is in the form of a tablet. In another embodiment, the article of manufacture further comprises a source of water. In yet another embodiment, the article of manufacture further comprises a second therapeutic gas.

In one embodiment, the article of manufacture is in the form of a bandage, wherein a substantially gas-tight cover is on one side of the first compartment, the second compartment is on the opposite side of the first compartment, and a rayon mesh on the side of the second compartment which is opposite first compartment. In another embodiment, an anti-bacterial agent is present on the same side of the second compartment as the rayon mesh.

The subject application provides for a bandage comprising: a) a substantially gas-tight cover; b) a first layer comprising a therapeutic gas storage form; c) a membrane; d) a second layer comprising a perfluorocarbon; and e) a rayon mesh, wherein the membrane separates the first and second layers. In a preferred embodiment, the therapeutic gas is oxygen. In another preferred embodiment, the therapeutic gas storage form is hydrogen peroxide.

In one embodiment, the therapeutic gas storage form is in the form of a tablet.

In one embodiment, the first layer further comprises a catalyst for releasing the therapeutic gas from the therapeutic gas storage form. In one embodiment, the catalyst provides control release of the therapeutic gas.

In another embodiment, the instant bandage is adapted to contain other therapeutic gases known to be beneficial by to those of ordinary skill in the art. The storage forms of these gases and the catalysts for releasing said gases from their corresponding storage forms are known to those of ordinary skill in the art.

In one embodiment, the first layer is encapsulated by a brittle material. In another embodiment, the bandage is activated by breaking the brittle material.

In one embodiment, the membrane is gas-permeable and liquid-impermeable. In another embodiment, the perfluorocarbon is perfluoro-tert-butylcyclohexane. In a preferred embodiment, the perfluorocarbon is Oxycyte®. In yet another embodiment, the rayon mesh further comprises an antimicrobial agent.

The subject application also provides for a bandage comprising: a) a substantially gas-permeable cover; b) a layer comprising a perfluorocarbon; and c) a rayon mesh.

In one embodiment, the perfluorocarbon is perfluoro-tert-butylcyclohexane. In a preferred embodiment, the perfluorocarbon is Oxycyte®. In another embodiment, the rayon mesh further comprises an antimicrobial agent.

The subject application also provides for a method of delivering oxygen to a tissue in a subject comprising administering to the tissue a composition comprising a perfluorocarbon and an oxygen storage form, wherein the perfluorocarbon and the oxygen storage form are separated by a gas-permeable and liquid-impermeable material, so as to thereby deliver oxygen to the tissue.

The subject application also provides for a method of delivering oxygen to a tissue in a subject comprising topically administering to the tissue a solid or semi-solid composition comprising a perfluorocarbon and an oxygen storage form, wherein the perfluorocarbon and the oxygen storage form are separated by gas-permeable and liquid-impermeable material, so as to thereby deliver oxygen to the tissue.

In one embodiment, the tissue is affected by a pathological condition. In another embodiment, the pathological condition is a wound. In another embodiment, the wound is a laceration, abrasion, graze, rupture, cut or puncture wound. In yet another embodiment, the wound is a burn wound. In yet another embodiment, the tissue is skin.

In one embodiment, the oxygen storage form is hydrogen peroxide, sodium peroxide, calcium peroxide, magnesium peroxide, zinc peroxide, lithium peroxide, urea hydrogen peroxide, sodium percarbonate, sodium percarbonate perhydrate, sodium carbonate perhydrate, sodium perborate, carbamide peroxide, histidine hydrogen peroxide, adenine hydrogen peroxide, anhydrous poly(vinyl pyrrolidone)/hydrogen peroxide complex, or alkaline peroxyhydrate including sodium orthopohsporate.

In one embodiment, the composition further comprises a catalyst for releasing oxygen from of the oxygen storage form. In another embodiment, the catalyst is a metal or metal alloy containing iron, copper, lead, platinum, silver, iodine, or mercury. In another embodiment, the catalyst is a metal oxide or metal salt including manganese dioxide, manganous oxide, titanium dioxide, ferric oxide, ferrous oxide, iron chloride, hydroxide of lead, silver cobalt, manganese, osmium, copper, nickel, iron, chromium, selenium and platinum. In another embodiment, the catalyst is an enzyme including catalase. In yet another embodiment, the catalyst is a hydrogen peroxide oxidizer including iodine, ferric iron compound, mercury compound, silver compound, inorganic nitrate, bromine, concentrated sulfuric acid, chlorine gas, chromate compound, permanganate compound, ozone, and fluorine. In one embodiment, the catalyst provides control release of oxygen.

In one embodiment, the perfluorocarbon is perfluoro-tert-butylcyclohexane. In a preferred embodiment, the perfluorocarbon is Oxycyte®.

In one embodiment, the therapeutic gas storage form is in the form of a tablet.

In one embodiment, the gas-permeable and liquid-impermeable material exhibits at least 10 inches of hydro-head. In another embodiment, the gas-permeable and liquid-impermeable material exhibits at least 30 inches of hydro-head. In yet another embodiment, the gas-permeable and liquid-impermeable material is composite fabric, spundbond-meltblown-spundbond fabric, spundbond-meltblown-spundbond laminate, hydrophobic coated paper, hydrophobic coated fabric, fiberglass filter, microporous polymer membrane, microporous sintered metal membrane, thermo-mechanically expanded polytetrafluoroethylene (PTFE), fluoropolymer, flashspun high-density polyethylene fiber, poly(glycolic acid), poly(vinyl alcohol), polymer coated fabric, fluoropolymer-based porous membrane, or polyurethane-coated fabric. In another embodiment, the gas-permeable and liquid-impermeable material is Gore-Tex®. In another embodiment, the gas-permeable and liquid-impermeable material is Teflon®-based porous membrane. In a preferred embodiment, the gas-permeable and liquid-impermeable material is Tyvek®.

In one embodiment, the composition is a pharmaceutical composition and comprises a pharmaceutically acceptable carrier. In another embodiment, the composition further comprises a second therapeutic gas. In yet another embodiment, the composition further comprises a pharmaceutically active compound.

In one embodiment, the composition further comprises a biologically active agent. In another embodiment, the biologically active agent is an antibacterial agent, copper oxide, antibiotic, phospholipid, collagen particle, anti-inflammatory agent, tissue regenerating compound, blood thinner, blood coagulant, pain reliever, anti-itch compound, anti-burn compound, analgesics, matrix metallo proteinase inhibitor, tetracycline, doxycycline, denatured collagen, gelatin, oxidized regenerated cellulose, honey, antifungal compound, NIMBUS antimicrobial compound, calcium alginate, hemostatic agent, protease inhibitor with broad specificity for the inhibition of serine, cysteine, aspartic proteases and amino peptidases, HIV, protein synthesis inhibitor, puromycin, anisomycin, Glyco Pore, Heta starch, alpha-1 proteinase inhibitor human, alpha-2-macroglobulin, tissue inhibitor of metalloproteinases, hyaluronic acid, glycoaminoglycan, proteoglycan, enzymatic debridement agent, bacterial collagenase, chemical or biogenerator for nitrous oxide, carbon dioxide, hydrogen sulfide or other therapeutic gases.

In one embodiment, the biologically active agent is a growth factor or cytokine. In another embodiment, the growth factor or cytokine is PDGF, KGF-2, TGF-β, bFGF, GM-CSF, heparin-binding growth factor-1, TGFα, VEGF, HIF-1, FGF, or CTGF.

In one embodiment, the biologically active agent is a protease inhibitor. In another embodiment, the protease inhibitor is amprenavir, fosamprenavir, indinavir, lopinavir, ritonavir, saquinavir, or nelfinavir.

In one embodiment, the composition is a perfluorocarbon emulsion. In another embodiment, the perfluorocarbon emulsion has a particle size of about 0.3 microns or less. In yet another embodiment, the perfluorocarbon emulsion has a particle size of about 0.05 to 0.1 microns.

In one embodiment, the composition is in the form of a bandage. In another embodiment, the composition is in the form of a gel. In yet another embodiment, the gel is a hydrogel.

In one embodiment, the composition is in the form of a scaffold. In another embodiment, the scaffold is produced by electrospinning. In yet another embodiment, the scaffold is implanted into the tissue.

In one embodiment, the composition is directly applied to the tissue. In another embodiment, the composition is biodegradable. In another embodiment, the composition is bioresorbable.

In one embodiment, the composition is used in conjunction with an oxygen delivery device. In another embodiment, the oxygen delivery device is an external membrane oxygenator.

In one embodiment, the subject is a mammal. In another embodiment, the mammal is human.

The subject application also provides for a method of delivering a therapeutic gas to a tissue in a subject comprising administering to the tissue a composition comprising a perfluorocarbon and a therapeutic gas storage form, wherein the therapeutic gas is nitric oxide, carbon monoxide, carbon dioxide, or hydrogen sulfide, so as to thereby deliver the therapeutic gas to the tissue.

The subject application also provides for a method of delivering a therapeutic gas to a tissue in a subject comprising topically administering to the tissue a solid or semi-solid composition comprising a perfluorocarbon and the therapeutic gas storage form, wherein the therapeutic gas is nitric oxide, carbon monoxide, carbon dioxide, or hydrogen sulfide, so as to thereby deliver the therapeutic gas to the tissue.

In one embodiment, the tissue is affected by a pathological condition. In another embodiment, the pathological condition is a wound. In another embodiment, the wound is a laceration, abrasion, graze, rupture, cut or puncture wound. In yet another embodiment, the wound is a burn wound. In another embodiment, the tissue is skin.

In one embodiment, the composition further comprises a catalyst for releasing the therapeutic gas from the therapeutic gas storage form. In one embodiment, the catalyst provides control release of the therapeutic gas.

In another embodiment, the instant method is adapted to deliver other therapeutic gases known to be beneficial by to those of ordinary skill in the art. The storage forms of these gases and the catalysts for releasing said gases from their corresponding storage forms are known to those of ordinary skill in the art.

In one embodiment, the perfluorocarbon is perfluoro-tert-butylcyclohexane. In a preferred embodiment, the perfluorocarbon is Oxycyte®.

In one embodiment, the therapeutic gas storage form is in the form of a tablet.

In one embodiment, the perfluorocarbon and a therapeutic gas storage form are separated by a gas-permeable and liquid-impermeable material. In another embodiment, the gas-permeable and liquid-impermeable material exhibits at least 10 inches of hydro-head. In another embodiment, the gas-permeable and liquid-impermeable material exhibits at least 30 inches of hydro-head. In yet another embodiment, the gas-permeable and liquid-impermeable material is composite fabric, spundbond-meltblown-spundbond fabric, spundbond-meltblown-spundbond laminate, hydrophobic coated paper, hydrophobic coated fabric, fiberglass filter, microporous polymer membrane, microporous sintered metal membrane, thermo-mechanically expanded polytetrafluoroethylene (PTFE), fluoropolymer, flashspun high-density polyethylene fiber, poly(glycolic acid), poly(vinyl alcohol), polymer coated fabric, fluoropolymer-based porous membrane, or polyurethane-coated fabric. In another embodiment, the gas-permeable and liquid-impermeable material is Gore-Tex®. In another embodiment, the gas-permeable and liquid-impermeable material is Teflon®-based porous membrane. In a preferred embodiment, the gas-permeable and liquid-impermeable material is Tyvek®.

In one embodiment, the composition is a pharmaceutical composition and comprises a pharmaceutically acceptable carrier. In another embodiment, the composition further comprises a second therapeutic gas. In another embodiment, the composition further comprises a pharmaceutically active compound.

In one embodiment, the composition further comprises a biologically active agent. In another embodiment, the biologically active agent is an antibacterial agent, copper oxide, antibiotic, phospholipid, collagen particle, anti-inflammatory agent, tissue regenerating compound, blood thinner, blood coagulant, pain reliever, anti-itch compound, anti-burn compound, analgesics, matrix metallo proteinase inhibitor, tetracycline, doxycycline, denatured collagen, gelatin, oxidized regenerated cellulose, honey, antifungal compound, NIMBUS antimicrobial compound, calcium alginate, hemostatic agent, protease inhibitor with broad specificity for the inhibition of serine, cysteine, aspartic proteases and amino peptidases, HIV, protein synthesis inhibitor, puromycin, anisomycin, Glyco Pore, Heta starch, alpha-1 proteinase inhibitor human, alpha-2-macroglobulin, tissue inhibitor of metalloproteinases, hyaluronic acid, glycoaminoglycan, proteoglycan, enzymatic debridement agent, bacterial collagenase, chemical or biogenerator for nitrous oxide, carbon dioxide, hydrogen sulfide or other therapeutic gases.

In one embodiment, the biologically active agent is a growth factor or cytokine. In another embodiment, the growth factor or cytokine is PDGF, KGF-2, TGF-β, bFGF, GM-CSF, heparin-binding growth factor-1, TGFα, VEGF, HIF-1, FGF, or CTGF.

In one embodiment, the biologically active agent is a protease inhibitor. In another embodiment, the protease inhibitor is amprenavir, fosamprenavir, indinavir, lopinavir, ritonavir, saquinavir, or nelfinavir.

In one embodiment, the composition is a perfluorocarbon emulsion. In another embodiment, the perfluorocarbon emulsion has a particle size of about 0.3 microns or less. In another embodiment, the perfluorocarbon emulsion has a particle size of about 0.05 to 0.1 microns.

In one embodiment, the composition is in the form of a bandage. In another embodiment, the composition is in the form of a gel. In yet another embodiment, the gel is a hydrogel.

In one embodiment, the composition is in the form of a scaffold. In another embodiment, the scaffold is produced by electrospinning. In yet another embodiment, the scaffold is implanted into the tissue.

In one embodiment, the composition is directly applied to the tissue. In another embodiment, the composition is biodegradable. In another embodiment, the composition is bioresorbable.

In one embodiment, the subject is a mammal. In another embodiment, the mammal is human.

Burn wound treatments are described in section 20, chapter 276, of The Merck Manual, 17^{th} Edition (1999), Merck Research Laboratories, Whitehouse Station, N.J., U.S.A. which is hereby incorporated by reference.

The biochemistry of wound healing and strategies for wound treatment is described Chin et al., (2007) “Biochemistry of Wound Healing in Wound Care Practice” Wound Care Practice, 2^{nd} ed., Best Publishing, AZ., which is hereby incorporated by reference.

The chemistry of oxygen generation by peroxide decomposition is described in PCT International Application Publication No. WO/2007/134304, which is hereby incorporated by reference.

### Terms

As used herein, and unless stated otherwise, each of the following terms shall have the definition set forth below.

“Accelerates healing” means an increased rate of wound repair and healing as compared to the rate of wound repair and healing in an untreated control subject.

“Administering to the subject” means the giving of, dispensing of, or application of medicines, drugs, or remedies to a subject to relieve or cure a pathological condition. Parenteral administration is one way of administering the instant compounds to the subject. Unless otherwise specified, administering to the subject does not include topical application. In the specific case of “topically administering to the subject” as used herein, the topical administration includes administration to the skin or an external mucosa membrane of a subject.

“Antibacterial agent” means a bactericidal compound such as silver nitrate solution, mafenide acetate, or silver sulfadiazine, or an antibiotic. According to the present invention, antibacterial agents can be present in “Curpon™” products. “Cupron™” products utilize the qualities of copper and binds copper to textile fibers, allowing for the production of woven, knitted and non-woven fabrics containing copper-impregnated fibers with the antimicrobial protection against microorganisms such as bacteria and fungi.

“Biologically active agent” means a substance which has a beneficial or adverse effect on living matters. According to the present invention, the biologically active agent can be an antibacterial agent, copper oxide, antibiotic, phospholipid, collagen particle, anti-inflammatory agent, tissue regenerating compound, blood thinner, blood coagulant, pain reliever, anti-itch compound, anti-burn compound, analgesics, matrix metallo proteinase inhibitor, tetracycline, doxycycline, denatured collagen, gelatin, oxidized regenerated cellulose, honey, antifungal compound, NIMBUS antimicrobial compound, calcium alginate, hemostatic agent, protease inhibitor with broad specificity for the inhibition of serine, cysteine, aspartic proteases and amino peptidases, HIV, protein synthesis inhibitor, puromycin, anisomycin, Glyco Pore, Heta starch, alpha-1 proteinase inhibitor human, alpha-2-macroglobulin, tissue inhibitor of metalloproteinases, hyaluronic acid, glycoaminoglycan, proteoglycan, enzymatic debridement agent, bacterial collagenase, chemical or biogenerator for nitrous oxide, carbon dioxide, hydrogen sulfide or other therapeutic gases. The biologically active agent can also be a growth factor or cytokine such as PDGF, KGF-2, TGF-β, bFGF, GM-CSF, heparin-binding growth factor-1, TGFα, VEGF, HIF-1, FGF, or CTGF. The biologically active agent can also be a protease inhibitor such as amprenavir, fosamprenavir, indinavir, lopinavir, ritonavir, saquinavir, or nelfinavir. In addition, freeze dried platelets, freeze dried serum, plasma and liver extract are some examples of sources of growth factors, cytokines, chemokines and clotting agents according to the present invention.

“Burn wound” means a wound resulting from a burn injury, which is a first, second or third degree injury caused by thermal heat, radiation, electric or chemical heat, for example as described at page 2434, section 20, chapter 276, of The Merck Manual, 17^{th} Edition (1999), Merck Research Laboratories, Whitehouse Station, N.J., U.S.A.

“Catalase” means the well-known catalase enzyme found in living organisms. Catalase catalyzes the decomposition of hydrogen peroxide to water and oxygen. This enzyme has one of the highest turnover rates for all enzymes; one molecule of catalase can convert millions of molecules of hydrogen peroxide to water and oxygen per second. The enzyme is a tetramer of four polypeptide chains, each over 500 amino acids long. It contains four porphyrin heme (iron) groups which allow the enzyme to react with the hydrogen peroxide. The optimum pH for catalase is approximately neutral (pH 7.0), while the optimum temperature varies by species. In the practice of the present invention, preparations of the enzyme, as are known in the art, may be utilized. Alternatively, in some embodiments, the use of a source of catalase, (e.g. a vector that encodes the enzyme, or an organism that is genetically engineered to overproduce the enzyme) may be appropriate. Furthermore, in some application agents other than catalase which are capable of liberating O₂ may be included.

“Effective” as in an amount effective to achieve an end means the quantity of a component that is sufficient to yield a desired therapeutic response without undue adverse side effects (such as toxicity, irritation, or allergic response) commensurate with a reasonable benefit/risk ratio when used in the manner of this disclosure. For example, an amount effective to promote burn wound healing without causing undue adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Electrospinning” means the process of manufacturing fibers which uses an electrical charge to draw very fine fibers from a liquid. The fibers may be microfibers or nanofibers.

“Hydro-head” is the pressure that must develop before a first drop of liquid is forced through a material. According to one embodiment of the present invention, a hydro-head of 30 inches of water means that the liquid on the therapeutic gas storage form side of the gas-permeable and liquid-impermeable material must be subjected to a pressure of greater than 30 inches of water in order to force a first drop of liquid through the material to the other side.

A “gas-permeable and liquid-impermeable” or “semi-permeable” material according to the present invention is a material which allows for the “free-flow” of a therapeutic gas but presents a “substantial barrier” to the passage of liquid. A material allows for the “free-flow” of a therapeutic gas when the material has sufficient permeability to the therapeutic gas to allow the surface of the PFC layer in the bandage according to the present invention to obtain efficaciously high concentrations of the therapeutic gas. The efficaciously high concentrations requirement depends on the severity of the wound and patient response to the gas therapy. This requirement is determined by experimentation and clinical usage but is always higher than concentration that would otherwise result from exposure to air. A “substantial barrier” to liquid flow means the material used exhibits a “hydro-head” of at least 10 inches of water, but preferably 30 inches of water of higher. According to a preferred embodiment of the present invention, Tyvek® is used as the “gas-permeable and liquid-impermeable” material. Because oxygen passes easily through Tyvek, the pressure developed internally by oxygen generation will be the same on both sides of the Tyvek. Thus, only mechanically applied pressure on the outer surface of the bandage can force liquid through the Tyvek. Other examples of materials that are suitable for use as the gas-permeable and liquid-impermeable material include but are not limited to: poly(lactic-co-glycolic acid) (PLGA) blends (e.g. pure polyglycolic acid (PGA), pure polylactic acid (PLA), and blends in the range of about 1:100 PGA to PLA or 1:100 PLA to PGA, or various blends with ratios in between e.g. about 10:90, 20:80, 30:70, 40:60 or 50:50, the composition being known to affect crystallinity and solubility and the transport rate of water and thus of H₂O₂; polyanhydrides; polysaccharides; polyamide esters; polyvinyl esters; polybutyric acid; poly(R)-3-hydroxybutyrate, poly(ε-caprolactones); etc. Since the invention is used to treat patients (humans or animals), the membrane material is preferably non-toxic and biodegradable. Exemplary biodegradable polymers for use in human and animal patients include without limitation poly(α-hydroxy esters) including poly(glycolicacid) polymers, poly(lactic acid) polymers, poly(lactic-co-glycolic acid) co-polymers, poly(ε-caprolactone) polymers, poly(ortho esters), polyanhydrides, Poly(3-hydroxybutyrate) copolymers, polyphosphazenes, fumarate based polymers including poly(propylene fumarate), poly(propylene fumarate co-ethylene glycol), and oligo(poly(ethylene glycol) fumarate), polydioxanones and polyoxalates, poly(amino acids), and pseudopoly(amino acids). Other suitable materials are composite fabric, spundbond-meltblown-spundbond fabric, spundbond-meltblown-spundbond laminate, hydrophobic coated paper, hydrophobic coated fabric, fiberglass filter, microporous polymer membrane, microporous sintered metal membrane, thermo-mechanically expanded polytetrafluoroethylene (PTFE), fluoropolymer, flashspun high-density polyethylene fiber, poly(glycolic acid), poly(vinyl alcohol), polymer coated fabric, fluoropolymer-based porous membrane, polyurethane-coated fabric, Gore-Tex®, or Teflon®-based porous membrane.

A “gas-tight” or “non-permeable” covering means a covering which prevents the free flow of gas. A “gas-permeable” covering, on the other hand, means a covering which allows the free flow of gas. According to the present invention, the “gas-tight” covering contains therapeutic gases and allows the concentration of said gas to build within the bandage structure thereby forcing the gas to move through the gas-permeable and liquid-impermeable membrane and subsequently to the wound. This definition encompasses materials that allow gas to diffuse through over a long time relative to the requirements of the bandage. Polymeric barriers such as polyethylene films, polypropylene films, polymer-coated papers and fabrics, and twin-ply constructions of polypropylene non-woven spundbond layer with waterproof polyethylene films can be used as the “gas-tight” covering according to the present invention. Although polyethylene films are technically permeable to oxygen over a time scale that could range from seconds to days depending on the thickness of the film, when designed properly, a polyethylene film will retain enough of the gas to force the migration of the gas onto the wound.

“Hydrogel” means any colloid in which the particles are in the external dispersion phase and water is in the internal dispersed phase.

“In the form of a tablet” means that the storage form is contained within a small solid aggregation of substances. According to the present invention, the therapeutic gas storage form, preferably hydrogen peroxide, may be supplied as a tablet. Preferably, the tablet is specifically compounded to control release hydrogen peroxide to the catalyst at the very slow rate required by the oxygen consumption rate expected from the wound. Control release of hydrogen peroxide and oxygen is desired to prevent waste. If hydrogen peroxide is readily and immediately released to the catalyst, most of the oxygen produced would be vented off and wasted because the wound would not be able to absorb and consume the all the oxygen produced. Similarly, the catalyst can be supplied as a tablet or otherwise fabricated to provide control release of the therapeutic gas to the wound.

“Liquid” means a fluid that has the particles loose and can freely form a distinct surface at the boundaries of its bulk material. The surface is a free surface where the liquid is not constrained by a container. Emulsions are specifically included in this definition of liquids.

“Novel Intrinsically Microbonded Utility Substrate” or “NIMBUS” is a technology that permanently bounds an antimicrobial polymer containing quaternary nitrogen groups to a wound dressing material. The bound antimicrobial polyquat prevents bacteria from penetrating to the surface of the wound, enhances absorption of wound exudates, and inhibits growth of bacteria in the dressing, which prevents shedding of large numbers of bacteria back onto the wound surface from a fouled dressing. In addition, the bound antimicrobial agent does not diffuse into the wound, thus avoiding the possibility of damaging wound cells and slowing healing.

“Oxygen color indicator” is a device which changes color upon exposure to oxygen, thereby determining the presence or absence of oxygen. An example of an oxygen color indicator is methylene blue, a dye widely used as a redox indicator. In its reduced state, it is colorless. In its oxidized state, it is a deep blue. A colorless solution of the reduced dye will turn blue upon exposure to air. An oxygen color indicator may also involve an electronic device.

“Oxygenated perfluorocarbon” is a perfluorocarbon which is carrying oxygen at, for example, saturation or sub-saturation levels.

“Pharmaceutically active compound” means the compound or compounds that are the active ingredients in a pharmaceutical formulation.

“Promotes alleviation of pain” means a decrease in the subject's experience of pain resulting from a wound.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention.

“Scaffold” means an artificial structure capable of supporting three-dimensional tissue formation.

“Solid or semi-solid composition” can take the form of the following non-limiting examples: cream, gel, hydrogel, oil, foam, wax, powder, paste, solid, scaffold, or aerosol spray. Liquids, including emulsions, are specifically excluded from the definition of a solid or semi-solid composition.

“Therapeutic gas storage form” means a non-gas in a suitable containment or compound comprising the atoms that constitute the gas, which under certain conditions, e.g., when reacted with a catalyst, produces the therapeutic gas at STP, i.e., standard temperature and pressure. The non-gas compound storage form is typically more stable and more amenable to processing than the gas itself. According to the present invention, the storage form of oxygen can be hydrogen peroxide, sodium peroxide, calcium peroxide, magnesium peroxide, zinc peroxide, lithium peroxide, urea hydrogen peroxide, sodium percarbonate, sodium percarbonate perhydrate, sodium carbonate perhydrate, sodium perborate, carbamide peroxide, histidine hydrogen peroxide, adenine hydrogen peroxide, anhydrous poly(vinyl pyrrolidone)/hydrogen peroxide complex, or alkaline peroxyhydrate including sodium orthopohsporate. The storage forms of other therapeutic gases are known by those of ordinary skill in the art.

According to the present invention, the catalyst for releasing oxygen from the oxygen storage form can be a metal or metal alloy containing iron, copper, lead, platinum, silver, iodine, or mercury; a metal oxide or metal salt including manganese dioxide, manganous oxide, titanium dioxide, ferric oxide, ferrous oxide, iron chloride, hydroxide of lead, silver cobalt, manganese, osmium, copper, nickel, iron, chromium, selenium and platinum; an enzyme including catalase; or a hydrogen peroxide oxidizer including iodine, ferric iron compound, mercury compound, silver compound, inorganic nitrate, bromine, concentrated sulfuric acid, chlorine gas, chromate compound, permanganate compound, ozone, and fluorine. The catalysts for releasing other therapeutic gases from their corresponding storage forms are known to those of ordinary skill in the art.

Perfluorocarbons (PFCs) possess the ability to dissolve large quantities of polar gases at concentrations much larger than water, saline and plasma. In addition, PFCs enhance the ability of these gases to diffuse across distances. Thus, PFCs can be a convenient and inexpensive means to deliver high levels of oxygen or other therapeutic gases to wounds and other organ systems.

Being that the PFCs are slightly lipophilic at body temperature and would help in the transport of oxygen into and removal of carbon dioxide from the skin tissue, PFCs can accelerate the healing process of a wound in a tissue. A preferred PFC, F-tert-butylcyclohexane, is only slightly lipophilic at body temperature and not lipophilic at room temperature.

PFCs that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of polar gases, both as emulsions for intravenous use and as neat liquids for liquid ventilation applications.

In one embodiment of the present invention, the PFC is perfluorodecalin. PFCs also include perfluoro-tert-butylcyclohexane (C₁₀F₂₀) which is available, for example, as Oxycyte® from Oxygen Biotherapeutics Inc., Costa Mesa, Calif. In an embodiment, the Perfluoro-tert-butylcyclohexane has the following structure:

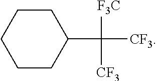

Oxycyte® is based on the perfluorocarbon F-tert-butylcyclohexane, a saturated alicyctic PFC (molecular formula C₁₀F₂₀) and can be used as a PFC composition in the methods and uses described herein. Physical properties of F-tert-butylcyclohexane are as follows:

Molecular Formula C₁₀F₂₀

Molecular Weight (g/mol) 500.08

Physical State @ Room Temp. Liquid

Density (g/mL) 1.97

Boiling Point (° C.) 147

Vapor Pressure (mmHg) @ 25° C. 3.8

Vapor Pressure (mmHg) @ 37° C. 4.4

Kinematic Viscosity (cP) 5.378

Refractive Index @ 20° C. 1.3098

Calculated Dipole Moment (Debye) 0.287

Calculated Surface Tension (dyne/cm) 14.4

The perfluorocarbon compositions may comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise the oxygenated perfluorocarbon. The composition employed in the methods described herein may also comprise a pharmaceutically acceptable additive.

PFCs are likely to lend themselves to processing using other biocompatible chemicals and processes to take on numerous forms. These include but are not limited to liquids, solids, semi-solids, gels, foams, etc. Incorporation into hydrogels and other delivery systems may allow for wound fluid absorption and conformance-adherence to the wound bed helping to assure a preferred level of wound moisture. Processes such as electrospinning may be used to make unique three dimensional scaffolds for wound healing. These forms may further be made to be biodegradable or bioresorbable. Thus PFCs may be made into numerous delivery devices for placement into wounds or tissues including simple direct application of PFCs into or onto wounds. PFCs when mixed with wound exudates may have similar properties as when they are mixed with plasma in that they enhance the diffusion of gases into the tissues.

Simple PFCs may allow for concentration of oxygen from atmospheric air to the surface of the wound with the PFC being the interface between the wound and the atmosphere. Furthermore it may be possible to enhance storage and delivery of oxygen to the wound with the assistance of other devices used in conjunction with PFC. This may include but is not limited to flowing oxygen or other oxygenated media over the wound with the wound being covered with oxygen. Furthermore exogenously oxygenated PFC could be streamed across the wound or tissue via an external membrane oxygenator. A number of devices now exist for placement over the wound that concentrate oxygen from the air into the wound. However, because of wound exudates, this high level of oxygen may be impeded for traversing the exudates since the exudates acts as a resistor to oxygen storage and diffusion.

These configurations may also be used to add other therapeutic gasses to the wound including but not limited to nitric oxide, carbon monoxide, carbon dioxide, hydrogen sulfide, and others. Additionally, storage forms or precursors of these gases may be used. Additional catalyst or chemicals may added to affect controlled production of these gases.

In a preferred embodiment, a PFC based gel or bandage or a combination thereof contains a stored form of oxygen such as hydrogen peroxide (H₂O₂), magnesium peroxide and calcium peroxide. Within the bandage are the necessary components which modulates the controlled conversion of the stored form of oxygen into oxygen which is then stored in the PFC and used by the wound as needed or at a set rate. Such configurations can be used as pastes over large burns, for example, or as dissolvable implants into large wounds and flaps which may have vascular compromise.

Similar embodiments are envisioned where other gases in their direct or precursor (storage) form are placed in the PFC for controlled delivery to the wound or tissue. Nitric oxide, carbon monoxide, and carbon dioxide are all known to be vasodialtors and to have other potential beneficial effects via their cell signaling properties. Hydrogen sulfide may have the potential to “suspend” wound metabolism. Even nitrogen may play a role. PFC allows these gases to be stored and transferred to a greater degree. Such configurations may be used as coverings or perfusates of organs awaiting transplant.

The multiplicity of configurations may contain additional beneficial active biological agents which further promote tissue health including but is not limited to growth factors, enzymatic debridement agents, hemostatics, and others. More complex configurations of tissue scaffolding such as one manufactured via electrospinning may contain PFCs with stored gases allowing for tissue coverings or tissue implants to provide oxygen to their surrounding damaged environment to promote healing or to activate and facilitate growth processes.

The perfluorocarbon emulsions of the methods of the invention include perfluorocarbon-in-water emulsions comprising a continuous aqueous phase and a discontinuous perfluorocarbon phase. The emulsions can include emulsifiers, buffers, osmotic agents, and electrolytes as well as the components described herein. The perfluorocarbons are present in the emulsion from about 5% to 130% w/v. Embodiments include at least about 40%, 45%, 50%, 55%, 60%, 65%, 70%, 75%, 80% and 85% w/v. A 60% w/v F-tert-butylcyclohexane emulsion may be used as the perfluorocarbon emulsion in one embodiment. Embodiments also include an egg yolk phospholipid emulsion buffered in an isotonic medium wherein the perfluorocarbon is present in the emulsion from about 5% to 130% w/v. Embodiments include at least about 40%, 45%, 50%, 55%, 60%, 65%, 70%, 75%, 80% and 85% w/v. A 60% w/v F-tert-butylcyclohexane emulsion may be used as the perfluorocarbon emulsion in one embodiment of an egg yolk phospholipid emulsion buffered in an isotonic medium.

The perfluorocarbons may be in a salt form. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compositions of this invention may be administered in various forms, including those detailed herein. The treatment with the compound may be a component of a combination therapy or an adjunct therapy, i.e. the subject or patient in need of the drug is treated or given another drug for the pathological condition in conjunction with one or more of the instant compounds. In one embodiment of the subject invention, PFCs are used in conjunction with a pharmaceutically active compound to treat a wound in the tissue. This combination therapy can be sequential therapy where the patient is treated first with one drug and then the other or the two drugs are given simultaneously. These can be administered independently by the same route or by two or more different routes of administration depending on the dosage forms employed. In an embodiment, a composition is provided comprising an amount of the compound effective to treat a pathological condition as specified above and a pharmaceutical carrier.

As used herein, a “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds also used to treat the pathological condition. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into the wounded tissue, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. Examples of suitable solid carriers include lactose, sucrose, gelatin and agar. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The PFCs can be administered parenterally, in sterile liquid dosage forms. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Parenteral and intravenous forms may also include minerals and other materials to make them compatible with the type of injection or delivery system chosen.

The instant compounds may also be administered via transdermal routes, using those forms of transdermal skin patches well known to those of ordinary skill in that art. To be administered in the form of a transdermal delivery system, the dosage administration will generally be continuous rather than intermittent throughout the dosage regiment.

The PFC compositions may contain the any of the following non-toxic auxiliary substances:

The PFC compositions may contain antibacterial components which are non-injurious in use, for example, thimerosal, benzalkonium chloride, methyl and propyl paraben, benzyldodecinium bromide, benzyl alcohol, or phenylethanol.

The PFC compositions may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The PFC compositions may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The PFC compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The PFC compositions may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The PFC compositions may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

It is understood that where a parameter range is provided, all integers within that range, and tenths thereof, are also provided by the invention. For example, “25-50%” includes 25.0%, 25.1%, 25.2%, 25.3%, 25.4% etc up to 50.0%. For example “10-20 mls/min” includes 10.0 mls/min, 10.1 mls/min, 10.2 mls/min, 10.3 mls/min etc. up to 20.0 mls/min.

In one particular embodiment of the present invention, the PFC composition is adapted for topical application as a bandage for wound healing. This bandage comprises a PFC composition, optionally in the form of a gel, and an oxygen storage form. The use of the PFC allows for the controlled production and release of oxygen since PFCs are capable of holding onto and transporting oxygen and other therapeutic gases. In addition, PFC facilitates oxygen dissolution into the wound and allows efficient delivery of oxygen into the wound. The present invention overcomes the drawbacks presented by previous designs. Hence, the bandage disclosed here is an improvement over the existing relevant art.

All combinations of the various elements are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Disclosed herein are methods using perfluorocarbon compositions as a supplier of therapeutic gases to and for the treatment of wounded tissues.

### Example 1

A composition comprising a perfluorocarbon and a therapeutic gas is administered to a tissue in a subject in need thereof.

The therapeutic gas is delivered to the tissue in the subject.

### Example 2

A solid or semi-solid composition comprising a perfluorocarbon and a therapeutic gas is topically administered to a tissue in a subject in need thereof.

The therapeutic gas is delivered to the tissue in the subject.

### Example 3

A composition comprising a perfluorocarbon is administered to a subject suffering from a wound in a tissue such that the composition forms an interface between the wound and the atmosphere.

The wound is treated. The wound shows accelerated healing.

### Example 4

### Bandage Efficacy Study

A bandage comprising a substantially gas-tight cover; a first layer comprising an oxygen storage form; a gas-permeable and liquid-impermeable membrane; a second layer comprising a perfluorocarbon; and a rayon mesh wherein the gas-permeable and liquid-impermeable membrane separates the first and second layers is administered to a wound in a subject. Tissue oxygenation is measured using Raman Spectroscopy.

Oxygen is delivered to the wound.

### Example 5

### Bandage Safety Study

A bandage comprising a substantially gas-tight cover; a first layer comprising an oxygen storage form; a gas-permeable and liquid-impermeable membrane; a second layer comprising a perfluorocarbon; and a rayon mesh wherein the gas-permeable and liquid-impermeable membrane separates the first and second layers is administered to a wound in a subject. A simple irritating intact skin study is conducted using Band-Aid as control. Bandage does not cause irritation.

A partial thickness wound human study is conducted with oxygenated Oxycyte® gel.

A NAMSA study is conducted.

Bandage is safe.

### Example 6

### Bandage Oxygen Permeability Study

A bandage comprising a substantially gas-tight cover; a first layer comprising an oxygen storage form; a gas-permeable and liquid-impermeable membrane; a second layer comprising a perfluorocarbon; and a rayon mesh wherein the gas-permeable and liquid-impermeable membrane separates the first and second layers is administered to a wound in a subject.

Oxygen is delivered to the wound.

### Example 7

### Bandage Shelf life Study

A shelf-life study for a bandage comprising a substantially gas-tight cover; a first layer comprising an oxygen storage form; a gas-permeable and liquid-impermeable membrane; a second layer comprising a perfluorocarbon; and a rayon mesh wherein the gas-permeable and liquid-impermeable membrane separates the first and second layers is conducted.

The bandage has a satisfactory shelf-life.

### Example 8

### Bandage Oxygen Release Study

A powdered mixture of urea hydrogen peroxide (UHP) powder (79.25 wt %), corn syrup solids (5.0 wt %), magnesium stearate (0.6 wt %), glutinous rice flour (5.0 wt %), and Methocel® K35 (10.15 wt %) was dried under vacuum at 40° C. for one hour and then fabricated into ¼″ diameter by 3/32″ thick tablets using an automated tablet press.

An oxygen-producing bandage was fabricated by lightly coating the normally outer surface of an extra large sheer bandage (CVS Pharmacy brand) with 5μ diameter manganese (II) dioxide. The surface of the bandage that covered an underlying gauze pad was lightly pre-coated with a spray adhesive (3M Company) to keep the manganese dioxide in place. A single UHP-containing tablet fabricated as described above was placed in the center of the manganese dioxide catalyst bed. The top of a second bandage was covered with a sheer film of polyurethane (Tegaderm®) as a gas barrier. The gauze pad of the second bandage was wetted with one milliliter of tap water and the second bandage was place over and adhered to the normally outer surface of the first bandage such that the wetted gauze of the second (top) bandage contacted the UHP-containing tablet adhered to the normally outer surface of the first (bottom) bandage.

The contact of water with the UHP-containing tablet caused the tablet to begin a slow swelling/dissolution process thereby exposing UHP powder to the water. The wetted UHP adduct split into urea and hydrogen peroxide thereby releasing hydrogen peroxide to the surrounding manganese dioxide catalyst bed. Immediately upon contact with the catalyst, hydrogen peroxide was decomposed into water and molecular oxygen. The oxygen gas then quickly permeated through the covering of the bottom bandage and was released through the underlying gauze pad.

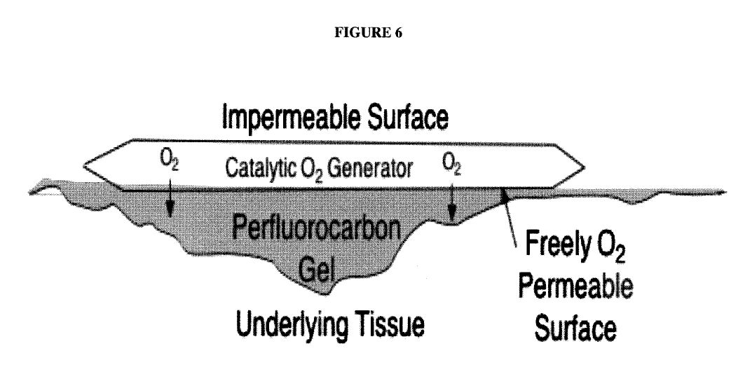

The rate of oxygen release was measured gravimetrically and found to be on the order of 2.2×10⁻⁴ g O₂/min. (FIG. 6) This rate is 5 times the rate considered to be effective as an aid to wound healing (4.4×10⁻⁵ g O₂/min). Lower or higher rates can be easily achieved by decreasing or increasing the tablet weight used.

1.-126. (canceled)

127. An article of manufacture comprising a therapeutic gas storage form and a perfluorocarbon, wherein when the therapeutic gas storage form is an oxygen storage form, it is in a first compartment and separated by a gas-permeable and liquid-impermeable material from the perfluorocarbon which is in a second compartment.

128. The article of manufacture of claim 127, wherein the oxygen storage form is hydrogen peroxide, or wherein the therapeutic gas is nitric oxide, carbon monoxide, carbon dioxide, or hydrogen sulfide.

129. The article of manufacture of claim 127, wherein the perfluorocarbon is perfluoro-tert-butylcyclohexane or wherein the therapeutic gas storage form is in the form of a tablet.

130. The article of manufacture of claim 127, wherein the therapeutic gas storage form is in a first compartment and the perfluorocarbon is in a second compartment.

131. The article of manufacture of claim 130, wherein the first and second compartments are separated by a gas-permeable and liquid-impermeable material.

132. The article of manufacture of claim 130, wherein the first compartment further comprises a catalyst for releasing the therapeutic gas from the therapeutic gas storage form, or wherein the article of manufacture further comprises a second different therapeutic gas.

133. The article of manufacture of claim 130, in the form of a bandage, wherein a substantially gas-tight cover is on one side of the first compartment, the second compartment is on the opposite side of the first compartment, and a rayon mesh is on the side of the second compartment which is opposite first compartment.

134. A bandage comprising:
i) a substantially gas-permeable cover;
ii) a layer comprising a perfluorocarbon; and
iii) a rayon mesh;
Or
a) a substantially gas-tight cover;
b) a first layer comprising a therapeutic gas storage form;
c) a membrane;
d) a second layer comprising a perfluorocarbon; and
e) a rayon mesh,
wherein the membrane separates the first and the second layers.

135. The bandage of claim 134, wherein the therapeutic gas is oxygen.

136. The bandage of claim 134, wherein the therapeutic gas storage form is hydrogen peroxide, or in the form of a tablet.

137. The bandage of claim 134, wherein the perfluorocarbon is perfluoro-tert-butylcyclohexane.

138. The bandage of claim 134, wherein the membrane is gas-permeable and liquid-impermeable.

139. The bandage of claim 134, wherein the first layer further comprises a catalyst for releasing the therapeutic gas from the therapeutic gas storage form.

140. A method of delivering a therapeutic gas to a tissue in a subject comprising administering or topically administering to the tissue a solid or semi-solid composition comprising a perfluorocarbon and a therapeutic gas storage form, wherein when the therapeutic gas storage form is an oxygen storage form, it is separated from the perfluorocarbon by a gas-permeable and liquid-impermeable material, so as to thereby deliver the therapeutic gas to the tissue.

141. The method of claim 140, wherein the oxygen storage form is hydrogen peroxide, or the therapeutic gas is nitric oxide, carbon monoxide, carbon dioxide, or hydrogen sulfide.

142. The method of claim 140, wherein the perfluorocarbon is perfluoro-tert-butylcyclohexane.

143. The method of claim 140, wherein the therapeutic gas storage form is in the form of a tablet.

144. The method of claim 140, wherein the perfluorocarbon and the therapeutic gas storage form are separated by a gas-permeable and liquid-impermeable material.

145. The method of claim 140, wherein the composition further comprises a catalyst for releasing the therapeutic gas from the therapeutic gas storage form, or a second different therapeutic gas.

146. The method of claim 140, wherein the composition is in the form of a bandage.
